(19) United States
(12) Plant Patent Application Publication
(10) Pub. No.: US 2006/0282924 P1
(43) Pub. Date: Dec. 14, 2006

(54) SEDUM PLANT NAMED 'MR. GOODBUD'

(50) SEDUM HYBRID; SEDUM

(75) Inventor: Gary J. Gossett, Portland, OR (US)

Correspondence Address: KLARQUIST SPARKMAN, LLP, 121 SW SALMON STREET, SUITE 1600, PORTLAND, OR, 97204, US

(73) Assignee: Terra Nova Nurseries, Inc.

(21) Appl. No.: 11/152,489

(22) Filed: Jun. 13, 2005

(51) Int. Cl. A01H 5/00

(52) U.S. Cl. PLT/263

(57) ABSTRACT

A new and distinct hybrid of Sedum plant characterized by red tinted stems and foliage, a compact, upright habit, large red purple and mauve flowers, and prominent white flower buds.

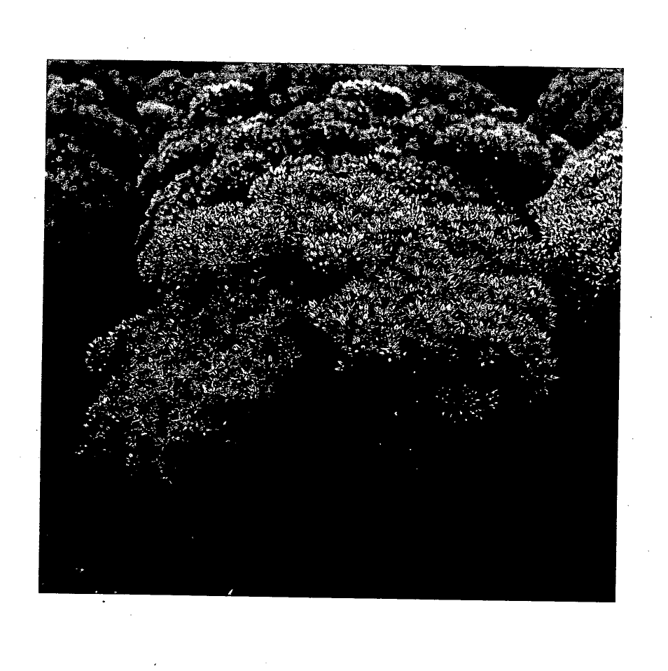

### BOTANICAL DENOMINATION

Sedum hybrid (S. spectabile ‘Brilliant’×S. telephium)

### VARIETY DESIGNATION

‘Mr. Goodbud’

The present invention relates to a new and distinct cultivar of Sedum hybrid and given the cultivar name of ‘Mr. Goodbud’. Sedum is in the family Crassulaceae. Sedum ‘Mr. Goodbud’ originated from an open pollinated cross between Sedum spectabile ‘Brilliant’ as the seed parent (unpatented) and Sedum telephium as the pollen parent (unpatented).

This new and unique Sedum is distinguished by:
1. Large red purple and mauve flowers.
2. Prominent, almost white flower buds
3. Red tinted stems and foliage.
4. Upright, compact growth habit.
5. Short stature.

This new cultivar has been reproduced only by asexual propagation (division and tissue culture). Each of the progeny exhibits identical characteristics to the original plant. Asexual propagation by division and tissue culture using standard micropropagation techniques with terminal and lateral shoots, as done in Canby, Oreg., shows that the foregoing characteristics and distinctions come true to form and are established and transmitted through succeeding propagations. The present invention has not been evaluated under all possible environmental conditions. The phenotype may vary with variations in environment without a change in the genotype of the plant.

### BRIEF DESCRIPTION OF THE DRAWING

The photograph shows Sedum ‘Mr. Goodbud’ growing in the ground in August in the trial fields in Canby, Oreg.

### DETAILED PLANT DESCRIPTION

The following is a detailed description of the new Sedum hybrid based on observations of one and a half year old specimen in September grown in the ground in full sun under typical outdoor conditions in the trial fields in Canby, Oreg. Canby is Zone 8 on the USDA Hardiness map. Temperatures range from a high of 95 degrees F. in August to an average of 32 degrees F. in January. Normal rainfall in Canby is 42.8 inches per year. The color descriptions are all based on The Royal Horticultural Society Colour Chart.
Botanical Classification: Sedum hybrid (S. spectabile ‘Brilliant’×S. telephium)
Variety Denomination: ‘Mr. Goodbud’
Plant:
   Type.—Herbaceous perennial.
   Hardiness.—USDA Zone 4 to 9.
   Size.—34 cm tall from the ground to the top of the foliage and 45 cm wide.
   Habit.—Compact, bushy.
   Vigor.—Excellent.
   Root description.—Multiple fleshy roots from each crown, Greyed Yellow 161C, grows to 5 mm wide and 20 cm long in clay loam soil.
Stem:
   Height.—66 cm.
   Width.—13 mm at base.
   Color.—Varies from Yellow Green 146B in the shaded areas and Greyed Purple 183C in the sun.
   Texture.—Succulent.
   Surface.—Glaucous.
Leaf:
   Type.—Simple.
   Arrangement.—Opposite.
   Shape.—Elliptic.
   Venation.—Pinnate.
   Margins.—Coarsely and irregularly serrate.
   Apex.—Broadly acute.
   Base.—Cuneate.
   Blade size.—Grows to 13 cm long and 4.5 cm wide.
   Surface.—Glaucous.
   Texture.—Thick, spongy, succulent.
   Petiole.—Leaves sessile.
   Leaf color.—Topside — Blue green (between Yellow Green 147A and Greyed Green 147A) tinged on sun side with maroon (Greyed Purple 187B). The color is variable depending on light and stress conditions. Leaves are darkest in full sun and low water. Bottom side — Between Yellow Green 147B and Greyed Green 147A.
Inflorescence:
   Type.—Terminal, corymbose, umbel-like.
   Size.—28 cm wide and 14 cm deep.
   Number of flowers.—up to 900 per main stem.
   Peduncle.—Height — 20 cm Diameter — 4 mm Color — Varies from Yellow Green 146B in the shaded areas and Greyed Purple 183C in the sun Texture — Succulent Surface — Glaucous.
   Branching.—up to 6 main branches on one stem.
   Bloom period.—Late August through September in Canby, Oreg.
Flower bud:
   Size.—2.5 mm wide, 6 mm deep.
   Description.—Ovoid.
   Color.—White 155A.
Flower:
   Type.—Complete, actinomorphic.
   Shape.—Stellate.
   Size.—9 mm deep and 15 mm wide
   Petal count.—5.
   Petal color.—Mauve, between Greyed Purple 186C and Red Purple 72B.
   Petal shape.—Lorate, reflexed.
   Petal size.—8 mm long, 1.5 mm wide
   Calyx.—Divided ⅘ to the base, campanulate, 3 mm deep and 2.5 mm wide.
   Calyx color.—Yellow Green 147B.
   Sepal number.—5 lobes.
   Stamen number.—5.
   Carpel.—Prominent, 5 separate.
   Pistil size.—6 mm deep and 1 mm wide.
   Pistil color.—Greyed Purple 186A to C.
   Stamens.—10, Greyed Purple 186B.
   Pollen color.—No pollen, male sterile.
   Fragrance.—None.
   Lastingness.—Each corymb lasts about five weeks.
Fruit:
   Type.—Ventrally dehiscent follicle, erect.
   Fertility.—Sterile.
   Color.—Brown 200A
Seed: No seed made
Pests and diseases: Sedum are susceptible to mealy bugs, scale insects, slugs and snails. No problems have been noted for this variety. All Sedum will develop root rot in wet conditions.
   Comparison to Similar Sedum

Compared to Sedum spectabile ‘Brilliant’, an unpatented plant similar and analogous to the mother parent, Sedum ‘Mr. Goodbud’ has darker foliage and stem and smaller inflorescences with duller pink flowers. They have similar habits. Sedum ‘Mr. Goodbud’ has strong stems that do not flop over after it blooms.

Compared to Sedum ‘Ruby Glow’, an unpatented plant, Sedum ‘Mr. Goodbud’ has a more upright habit and has noticeable, white, flower buds.

I claim:

1. A new and distinct hybrid of Sedum plant as herein illustrated and described.
